(19) United States
(12) Patent Application Publication
(10) Pub. No.: US 2025/0009687 A1
(43) Pub. Date: Jan. 9, 2025

(54) COMPOSITIONS AND METHODS USING A COMBINATION OF NUTRIENTS TO SUPPORT COGNITION AND EMOTIONAL HEALTH IN A MAMMAL

(71) Applicant: SOCIETE DES PRODUITS NESTLE S.A., Vevey (CH)

(72) Inventors: LAURA TROVO, Paudex (CH); PASCAL STEINER, St. Louis, MO (US); YUANLONG PAN, Chesterfield, MO (US); NICOLAS PREITNER, Lausanne (CH)

(21) Appl. No.: 18/705,413

(22) PCT Filed: Oct. 27, 2022
(86) PCT No.: PCT/EP2022/080035

§ 371 Date: Apr. 26, 2024

Related U.S. Application Data
(60) Provisional application No. 63/272,958, filed on Oct. 28, 2021.
(60) Provisional application No. 63/275,690, filed on Nov. 4, 2021.

(51) Int. Cl. A61K 31/132; A61K 31/12; A61K 31/202; A61K 31/37

(52) U.S. Cl. CPC A61K 31/132; A61K 31/12; A61K 31/202; A61K 31/37

(57) ABSTRACT

Compositions and methods can use at least one of (a) a combination of spermidine and ellagic acid or (b) a combination of docosahexaenoic acid (DHA) and curcumin to enhance cognition and emotional health in a mammal. The mammal is preferably an ageing or elderly human or an ageing or elderly companion animal, preferably an elderly dog.

### BACKGROUND

The present disclosure generally relates to to mammalian nutrition and effects thereof on cognitive function, behavior, and brain physiology, and also emotional health. In particular, the present disclosure utilizes a combination of nutrients, such as a combination of spermidine and ellagic acid, and/or a combination of docosahexaenoic acid (DHA) and curcumin, to support cognition in a mammal, such as an aging or elderly mammal, for example a human or a companion animal, as well as emotional control of their thoughts, feelings, and behaviors.

Aged or aging animals frequently suffer some degree of cognitive impairment. Changes, including decline in cognitive function that progresses with age, and age-related changes in brain morphology and cerebrovascular function are commonly observed, e.g., brain aging. Age-related or age-associated cognitive impairment may manifest itself in many ways, e.g., short-term memory loss, diminished capacity to learn, diminished rate of learning, diminished attention, diminished motor performance, and/or dementia, among other indicia. In some cases, a specific etiology of such cognitive decline is unknown. In other cases, cognitive impairment results from the onset or progression of recognized diseases, disorders, or syndromes, for example, Alzheimer's disease (AD). It is known that age-associated cognitive decline is distinct from and can occur independently of AD.

Animal models of cognitive impairment greatly facilitate the study of such conditions including their physiology, neurology, anatomy, and pathology. Dogs are useful model animals that demonstrate age-associated cognitive decline in learning and memory that varies depending on the function of the cognitive task (Adams B et al., 2000a; Chan A D F et al., 2002; Su M-Y et al., 1998; and, Tapp P D et al., 2003). While the study of such decline in dogs is useful in its own right because of their role as companion animals, the fact that the observed decline mirrors age-related cognitive declines seen in humans (Adams B et al. 2000b) makes the studies even more valuable. Aged dogs develop neuropathology that is related to that seen in both successfully aging humans and patients with AD, such as beta amyloid protein (Cotman C W and Berchtold, 2002; and Cummings B J et al., 1996). However, dogs do not demonstrate every hallmark of AD, in particular, tau-containing neurofibrillar tangles (Dimakopoulos A C et al., 2002) have not been observed. Therefore, the condition in dogs is distinct and referred to as Canine Cognitive Dysfunction Syndrome (CCDS).

Both healthy dogs and unhealthy dogs such as those diagnosed with CCDS may present clinically with progressive cognitive impairment and neuropathological changes (London E D et al., 1983). In addition, aging dogs and those diagnosed with CCDS exhibit various behavioral disorders. For example, they may not respond to their name or familiar commands, may get lost or confused even in familiar surroundings, may no longer greet or respond to their owners or visitors, may exhibit diminished daytime activity, may walk in circles, may shun affection, and may lose bladder or bowel control.

The senior companion animal is the fastest growing segment of the pet population. Importantly, senior pets experience behavioral changes that are linked to brain deficits, such as anxiety, aggressivity and cognitive impairment. These intermingled behavioral changes have a measurable and perceived impact on the emotional wellbeing of the pet and the owner.

### SUMMARY

Dietary modifications are powerful interventions to delay and slow the progression of many aging-related diseases, including brain-related impairments, and consumers are more and more aware of this notion. The most effective and scientifically validated interventions that are beneficial during ageing are based on either caloric restriction, a dietary regimen that reduces food intake without incurring malnutrition, or the implementation of fasting regimens.

However, caloric restriction and fasting are accompanied by significant nutrition and behavior constraints and lead for most people to low adherence to such dietary protocols or resistance to drastic changes to lifestyle, and these challenges are also relevant for pets.

Furthermore, existing data about improving mental and cognitive health by activating autophagy currently focuses on lifestyle changes, such as caloric restriction, intermittent fasting, or long-term exercise. These lifestyles are not easy to comply with, especially for middle-aged and senior people, making the solution ineffective for most of the population. At the same time, supplementation with single nutrients may require high doses or may have low efficacy, opening the need to discover more efficacious combinations.

Therefore, the present inventors sought to find a nutritional solution or nutrients that act as “caloric restriction mimetics (CRMs).” CRMs are defined as ingredients that simulate the health-promoting effects of fasting without the need of life-style changes. A list of known and putative CRMs in animal models and humans has been recently published in Madeo at al. Cell Metab. 20191.

The present disclosure leverages the very well-validated science linked to CRMs to develop a nutritional solution that will first support cognitive performance and mood while aging (cognition, anxiety, social behaviours), and second, improve significantly the quality of life of senior pets and indirectly of their owners, for example senior dogs aged seven years or more.

Complementary to the behavioral benefits, the “biomarker signature” of the blend is shown in a rodent study. Biomarkers could include blood glucose/insulin metabolism, blood oxidative stress markers (e.g., C-reactive proteins), and delayed biologic aging as measured by epigenetics, as well as inflammatory markers. Non-invasive biomarkers can then be used in a dog study and for communication to veterinary practitioners.

As set forth herein, the present inventors found a synergistic activation of Transcription Factor EB (TFEB) downstream genes involved in the lysosome biogenesis of similar effect size than other solutions reported in literature. This effect should increase the efficacy to support cognitive and emotional health at the same time.

Relative to single ingredient supplementation, the present disclosure provides more efficient and synergistic blends of nutrients to support cognition and emotional health (e.g., anxiety, mood and despair). It fills the gap between lifestyle changes and efficacious nutritional solutions to support cognition and emotional state in the adult and senior pets and humans.

Accordingly, in a general embodiment, the present disclosure provides compositions comprising a combination of nutrients for enhancing cognitive function and emotional health. The present disclosure also provides methods comprising administering one of these compositions in an amount effective for enhancing cognitive function and emotional health. The enhancement of cognitive function and emotional health can comprise one or more of (a) preventing, reducing, delaying and/or treating at least one of cognitive impairment, neurodegeneration conditions, stroke, dementia or age-related cognitive-decline; (b) decreasing at least one of anxiety, depression, negative emotions stress, aggressivity, despair, lack of focus, or irritability; and/or (c) maintaining or improving at least one of mood, happiness, positive emotions, mental wellbeing or social behavior (e.g., decreasing impulsive behavior).

The composition is administered to a mammal (e.g., a mammal in need of enhaced cognition and emotional health), preferably a companion animal or a human, for example an aging companion animal, an elderly companion animal, an aging human or an elderly human.

In some embodiments, the combination of nutrients comprises a combination of spermidine and ellagic acid. In some embodiments, the combination of nutrients comprises a combination of docosahexaenoic acid (DHA) and curcumin.

An advantage of one or more embodiments provided by the present disclosure is compositions and methods that improve cognition and emotional health, particularly in companion animals or humans, such as aging companion animals or aging humans.

Another advantage of one or more embodiments provided by the present disclosure is compositions and methods for treatment and/or prevention of cognitive impairment, neurodegeneration conditions, stroke, and dementia.

Still another advantage of one or more embodiments provided by the present disclosure is to improve the overall quality of life for both pets and pet owners.

An additional advantage of the present disclosure is to improve not only the lifespan of individuals and their pets, but their quality of life.

Additional features and advantages are described in, and will be apparent from, the following Detailed Description and the Figures.

### BRIEF DESCRIPTION OF DRAWINGS

FIGS. 1A and 1B show results regarding autophagy-related genes from a combination of DHA and curcumin in the experimental example disclosed herein.

FIGS. 2A and 2B show results regarding lysosomes-related genes from a combination of DHA and curcumin in the experimental example disclosed herein.

FIGS. 3A and 3B show results regarding autophagy-related genes from a combination of spermidine and ellagic acid in the experimental example disclosed herein.

FIGS. 4A and 4B show results regarding lysosomes-related genes from a combination of spermidine and ellagic acid in the experimental example disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal capable of autophagy. As used herein, the term “patient” is understood to include an animal, for example a mammal, and preferably a human that is receiving or intended to receive treatment, as treatment is herein defined. While the terms “individual” and “patient” are often used herein to refer to a human, the present disclosure is not so limited.

Accordingly, the terms “individual” and “patient” refer to any animal, mammal or human that can benefit from the methods and compositions disclosed herein. Indeed, non-human animals undergo prolonged critical illness that mimics the human condition and experience the effects of ageing as well.

The term “elderly” in the context of a human means an age from birth of at least 55 years, preferably above 63 years, more preferably above 65 years, and most preferably above 70 years. The term “older adult” or “ageing individual” in the context of a human means an age from birth of at least 45 years, preferably above 50 years, more preferably above 55 years, and includes elderly individuals.

For other animals, such as a companion animal (i.e., cat or dog), an “older adult” or “ageing individual” has exceeded 50% of the average lifespan for its particular species and/or breed within a species. An animal is considered “elderly” or “senior” if it has surpassed 66% of the average expected lifespan, preferably if it has surpassed the 75% of the average expected lifespan, more preferably if it has surpassed 80% of the average expected lifespan. An ageing cat or dog has an age from birth of at least about 5 years. An elderly or senior cat or dog has an age from birth of at least about 7 years.

The term “cognitive function” refers to the special, normal, or proper physiologic activity of the brain, including one or more of the following: mental stability, memory/recall abilities, problem solving abilities, reasoning abilities, thinking abilities, judging abilities, ability to discriminate or make choices, capacity for learning, ease of learning, perception, intuition, attention, and awareness. “Enhanced cognitive function” or “improved cognitive function” refers to any improvement in the special, normal, or proper physiologic activity of the brain, including one or more of the following: mental stability, memory/recall abilities, problem solving abilities, reasoning abilities, thinking abilities, judging abilities, ability to discriminate or make choices, capacity for learning, ease of learning, perception, intuition, attention, and awareness, as measured by any means suitable in the art.

Further in this regard, cognition is the mental process of learning and understanding through thought, experience, and the senses. Cognition includes perception; attention; the formation of knowledge, memory, judgment and evaluation; reasoning, problem solving and decision making; and comprehension and creation of language.

As non-limiting examples, cognitive function can be assessed by different cognitive tasks, specified per cognitive domain, although the various embodiments of the compositions and methods disclosed herein are not limited to specific tests.

For example, the cognitive domain of executive function (e.g., concept formulation, abstraction, set shifting, set maintenance, planning, self-monitoring, divided attention) can be assessed by common neurocognitive tests such as Wisconsin Card Sorting Test (WCST), Trail-Making Test Part B (TMT B), Stroop Colour-Word Interference Test (SCWT), Categories Test, Block Design (WAIS-R), Picture Completion (WAIS-R), Concept Shifting Task (CST), Tower of London (TOL; CANTAB), Stockings of Cambridge (SOC; CANTAB), Intra/Extradimensional Shift Test (CANTAB; IED), Spatial Span (SSP; CANTAB), Ruff Figural Fluency Test (RFFT), Verbal Fluency-Letter Fluency & Category Fluency, Controlled Oral Word Association Test (COWAT), and The Delis-Kaplan Executive Function System (D-KEFS).

As another example, the cognitive domain of attention and processing speed can be assessed by one or more common neurocognitive tests such as Digit Symbol Substitution Test (DSST; WAIS-R), Digit Span Forwards and Backwards (WAIS-R), Continuous Performance Task (CPT), Reaction Time (RTI; CANTAB), Choice Reaction Time (CRT; CANTAB), Simple Reaction Time (SRT; CANTAB), Trail-Making Test Part A (TMT A), Paced Auditory Serial Addition Test (PASAT), and Serial Sevens Subtraction Test (SSST).

As yet another example, the cognitive domain of working memory can be assessed by one or more common neurocognitive tests such as arithmetic (WAIS-R), Digit Span Forwards and Backwards (WAIS-R), Delayed Recognition Span Test (DRST), Spatial Working Memory (SWM; CANTAB), Letter-Number Sequencing (LNS; WMS-R), Logical Memory (WMS-R), and n-Back Test.

For example, the cognitive domain of verbal learning and memory can be assessed by one or more common neurocognitive tests such as California Verbal Learning Test (CVLT), Rey Auditory Verbal Learning Test (RAVLT), Rivermead Behavioral Memory Test (RBMT), Hopkins Verbal Learning Test Revised (HVLT-R), Logical Memory (WMS-R), Verbal Paired Associates (VPA; WMS-R), Visual Verbal Learning Test (VVLT), Digit Span Forwards and Backwards (WAIS-R), Luria Verbal Learning Test (LVLT), Serial Sevens Subtraction Test (SSST), and Verbal Recognition Memory Test (VRM; CANTAB).

As another example, the cognitive domain of visual learning and memory can be assessed by one or more common neurocognitive tests such as Visual Reproduction (WMS-R), Benton Visual Retention Test (VRT), Benton Visual Form Discrimination (VFD), Rey-Osterrieth Complex Figure Test (ROCF), Kimura's Recurring Figures Test (RFT), Visual Verbal Learning Test (VVLT), Pattern Recognition Memory (PRM; CANTAB), Spatial Recognition Memory (SRM; CANTAB), Delayed Matching to Sample (DMS; CANTAB), Paired Associates Learning (PAL; CANTAB), and Matching Familiar Figures Test 2 (MFFT-20).

As yet another example, the cognitive domain of language and verbal comprehension can be assessed by one or more common neurocognitive tests such as Controlled Oral Word Association Test (COWAT), Verbal Fluency-Category Fluency & Letter Fluency, Similarities (WAIS-R), Vocabulary (WAIS-R), Information (WAIS-R), Comprehension (WAIS-R), and Token Test.

For example, the cognitive domain of visuospatial/perceptual processing can be assessed by one or more common neurocognitive tests such as Judgement of Line Orientation (JOLO), Benton Visual Form Discrimination (VFD), Block Design (WAIS-R), and Visuospatial Span Forwards and Backwards (WMS-R).

As another example, the cognitive domain of brief mental status and general intelligence can be assessed by one or more common neurocognitive tests such as Mini Mental State Exam (MMSE), Raven's Progressive Matrices, Wechsler Adult Intelligence Scale-Revised (WAIS-R), National Adult Reading Test (NART), Wechsler Test of Adult Reading (WTAR), and Test of Nonverbal Intelligence-3 (TONI-3).

As yet another example, the cognitive domain of cognitive battery can be assessed by one or more common neurocognitive tests such as Cambridge Neuropsychological Test Automated Battery (CANTAB), Wechsler Memory Scale Revised (WMS-R), Wechsler Adult Intelligence Scale-Revised (WAIS-R), Victoria Symptom Validity Test (VSVT), California Computerised Assessment Package (CalCAP), CNS Vital Signs, Massachusetts General Hospital Cognitive and Physical Functioning Questionnaire (CPFQ), and The Delis-Kaplan Executive Function System (D-KEFS).

For example, the cognitive domain of psychomotor performance can be assessed by one or more common neurocognitive tests such as Finger Tapping, Grooved Pegboard Test, and Purdue Pegs.

As another example, the cognitive domain of decision making and response control can be assessed by one or more common neurocognitive tests such as The Go/No-go Association Task (GNAT; CANTAB), Information Sampling Task (IST; CANTAB), and Cambridge Gambling.

As another example, the cognitive domain of induction can be assessed by one or more common neurocognitive tests such as Big/Little Circle (BLC; CANTAB).

As used herein, “emotional health” can be assessed, for example, by one or more of: clinical interviews, behavior or symptom checklists, self-report measures of personality, states, traits and behavior, and projective tests (e.g., drawings, sentence completion, story telling, inkblot) or physiological markers linked to emotional responses and regulation (e.g. autonomic nervous system-related markers like heart rate, hear rate variability, breathing rate, skin conductance; e.g. central nervous system-related: structure, function or activation of mood-, stress- or emotion-regulation-related brain regions and networks like amygdala, hippocampus, prefrontal coretex, emotion regulation network; EEG and event-related potentials e.g. linked to emotion recognition etc.). The term “behavior” means anything that an animal does in response or reaction to a given stimulation or set of conditions. “Enhanced behavior” or “improved behavior” means any improvement in anything that an animal does in response or reaction to a given stimulation or set of conditions. “Behavior” is used synonymously with “behavioral function” herein.

Further in this regard, emotional health is about the sense of wellbeing and the ability to cope with real life events. People and animals with good emotional health are able to manage their thoughts, feelings, and behaviors properly, cope with real life's challenges well, are mostly in good spirits, and maintain good social interactions.

Signs of poor emotional health include feeling sad or down, having confused thinking, showing reduced ability to concentrate, having excessive fears or worries, or extreme feelings of guilt, showing extreme mood changes of highs and lows, withdrawal from friends (pets) and activities, feeling significant tiredness or low energy, having problems in sleeping.

The terms “condition” and “disorder” mean any disease, condition, symptom, or indication. “Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a pet owner, a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition comprising a combination of nutrients as disclosed herein (e.g., a combination of spermidine and ellagic acid, or a combination of DHA and curcumin) relative to a composition without the particular combination of ingredients but otherwise identical.

The terms “food,” “food product” and “food composition” mean a product or composition that is intended for ingestion by an individual such as a companion animal or human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

As used herein, “complete nutrition” contains sufficient types and levels of macronutrients (protein, fats and carbohydrates) and micronutrients to be sufficient to be a sole source of nutrition for the animal to which the composition is administered. Individuals can receive 100% of their nutritional requirements from such complete nutritional compositions.

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

### EMBODIMENTS

An aspect of the present disclosure is a composition comprising a combination of nutrients, such as a combination of spermidine and ellagic acid, or a combination of docosahexaenoic acid (DHA) and curcumin, in an effective amount to support or enhace cognition and emotional health in a mammal (e.g., emotional control of their thoughts, feelings, and behaviors). For example, in some embodiments, a unit dosage form comprises an amount of a combination of nutrients, such as a combination of spermidine and ellagic acid, or a combination of docosahexaenoic acid (DHA) and curcumin, in an effective amount to support or enhace cognition and emotional health in a mammal.

Another aspect of the present disclosure is a method for supporting or enhancing cognition and emotional health in a mammal (e.g., a mammal in need of enhaced cognition and emotional health), for example an aging mammal, such as a human or a companion animal (e.g., enhancing emotional control of their thoughts, feelings, and behaviors). The method comprises the steps of: (1) identifying an aging mammal having, or at risk of, decline in cognitive function and/or emotional health and (2) administering to the mammal an effective amount of a combination of nutrients, such as a combination of spermidine and ellagic acid, or a combination of docosahexaenoic acid (DHA) and curcumin.

Preferably the method is performed without caloric restriction. For example, the composition can be administered to the mammal at least once per week, preferably all seven days of the week, for a time period of at least one week, preferably at least one month, and the daily caloric intake during the time period of administration is substantially the same as that of the preceding week, preferably that of the preceding month.

Wheat germ is rich in spermidine. Therefore, some embodiments of the composition comprise wheat germ and/or enriched wheat germ extract that comprises at least a portion of the spermidine in the composition.

Ellagic acid is a phenolic acid. Some embodiments of the composition comprise pomegranate and/or enriched pomegranate extract that comprises at least a portion of the ellagic acid in the composition.

Docosahexaenoic acid (DHA) is C22:6n-3. At least a portion of the DHA in the composition can be in free form (as a fatty acid or a physiologically acceptable salt thereof) or comprised in a fatty acid derivative structure. In some embodiments, the composition comprises triglycerides comprising at least a portion of the DHA. DHA is present in natural sources, such as, for example, egg, algae or fish oil.

Curcumin is the polyphenol 1,7-bis(4-hydroxy-3-methoxyphenyl)hepta-1,6-diene-3,5-dione.

Non-limiting examples of concentrations and/or daily doses of these compounds follow hereafter.

For curcumin administered to pets, 0.25 mg to 250 mg of curcumin per kg body weight of the animal per day is suitable in various embodiments of the methods and compositions disclosed herein. In one aspect, 0.5 mg to about 100 mg of the curcumin per kg body weight of the animal can be administered per day. The curcumin is preferably present in an amount of 0.00025% to 2% of the food, preferably 0.0005% to 1% of the food.

For curcumin administered to humans, the curcumin may be present in amount of about 0.01 mg to about 2.0 g per serving, preferably from about 0.1 mg to about 2.0 g per serving, even more preferably from about 10.0 mg to about 1.0 g per serving.

For humans, particularly preferred phenols are rosmarinic acid, ellagic acid, and cholorgenic acid, or any possible combination or mixtures thereof. Each of the phenols or the sum of a combination of those phenols (or in the form of extracts) can be present in the composition, or in a composition that has been reconstituted from powder or concentrated form, at a concentration between 0.1% and 1%, between 0.15% and 0.8%, between 0.15% and 0.7%, between 0.15% and 0.6%, between 0.15% and 0.5%, between 0.2% and 0.4%, or be present at a concentration of about 0.3% in the composition.

For ellagic acid administered to pets, a suitable dose in various embodiments of the methods and compositions disclosed herein is 0.1 mg to 1000 mg per kg body weight of the animal per day. In one aspect, 1 mg to about 500 mg of the ellagic acid per kg body weight of the animal can be administered per day. In food, ellagic acid is preferably present at 0.1 mg/kg to 10,000 mg/kg of the food, more preferably 10 mg/kg to 1000 mg/kg of the food, and most preferably 100 mg/kg to 750 mg/kg of the food.

Non-limiting but particularly preferred doses of ellagic acid for a human include a daily dose of 50 mg-200 mg and 0.83 mg-3.33 mg per kg body weight of the subject. Non-limiting but particularly preferred doses of ellagic acid for a dog include a daily dose of 15 mg-60 mg (e.g., for a 10 kg dog) and 1.5 mg-6 mg per kg body weight of the dog. Examples sources of ellagic acid include 2 g-8 g of raw chestnuts and 87 mL-350 mL pomegranate juice.

For DHA administered to pets, a suitable dose in various embodiments of the methods and compositions disclosed herein is 1 mg to 1000 mg of DHA per kg body weight of the animal per day. In one aspect, 5 mg to about 750 mg of the DHA per kg body weight of the animal can be administered per day. The DHA is preferably present in an amount of 0.005% to 2% of the food, preferably 0.05% to 2% of the food.

For humans, a preferred embodiment of the daily amount of the omega-3 fatty acid (e.g., DHA) is about 50 mg to about 5 g of the omega-3 fatty acid per day, preferably about 100 mg to about 5 g of the omega-3 fatty acid per day, more preferably about 250 mg to 2.5 g omega-3 fatty acid per day, and most preferably about 0.5 g to about 1.5 g omega-3 fatty acid per day.

For pets, some embodiments of the methods and compositions disclosed herein include administering a daily dose of the autophagy inducer (e.g., spermidine) in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight. Typically between 50 μg to 10 g of the autophagy inducer, preferably a spermidine compound, can be administered per daily dose in one or more portions.

For spermidine administered to humans, a suitable dose in various embodiments of the methods and compositions disclosed herein is 0.09 mg to 50 mg per kg body weight of the animal per day, In one aspect, 0.1 mg to about 25 mg of the spermidine per kg body weight of the animal can be administered per day. In food, spermidine is preferably present at 0.1 mg/kg to 10.00 mg/kg of the food, more preferably 10 mg/kg to 500 mg/kg, and most preferably 25 mg/kg to 250 mg/kg of the food.

The composition can be formulated for consumption by any mammal. In certain embodiments, the mammal is a non-human, and in specific embodiments the mammal is a companion animal. Exemplary embodiments feature compositions formulated for consumption by a pet such as a companion animal, e.g., a dog or cat. In other embodiments, the mammal is a human. The composition administered to the mammal can be a pet food, dietary supplement, or a food product formulated for human consumption.

The composition may be formulated for administration to a healthy aging mammal. In certain embodiments, the mammal has a phenotype associated with age-related cognitive impairment. Such a phenotype can include one or more of decreased ability to recall, short-term memory loss, decreased learning rate, decreased capacity for learning, decreased problem solving skills, decreased attention span, decreased motor performance, increased confusion, or dementia, as compared to a control mammal not having the phenotype.

The composition can be administered on a regular basis, which in some embodiment, is at least once daily. In certain embodiments, the composition is administered as part of a daily dietary regimen for at least about one week, or at least about one month, or at least about three months up to at least about a year longer, extending to the duration of the mammal's life. The therapeutically effective dose can be determined by the person skilled in the art and will depend on a number of factors known to those of skill in the art, such as the severity of the condition and the weight and general state of the individual.

The composition is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved. In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

The term “long-term administration” means periods of repeated administration or consumption in excess of one month. Periods of longer than two, three, or four months are preferred for certain embodiments. Also preferred are more extended periods that include longer than 5, 6, 7, 8, 9, or 10 months. Periods in excess of 11 months or 1 year are also preferred. Longer term use extending over 1, 2, 3, or more years are included in the present disclosure. For certain aging animals, the animal will continue consuming on a regular basis for the remainder of its life. Sometimes this is referred to as consumption for “extended” periods.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, for example at least one component separately from one or more of the other components of the composition.

In various embodiments, pet food or pet treat compositions comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the composition ranges from less than 1% to about 15%, preferably from about 5% to about 10%.

The moisture content can vary depending on the nature of the composition. In a preferred embodiment, the composition is a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one presently preferred embodiment, the compositions have moisture content from about 5% to about 20%. Dry food products include a variety of foods of various moisture contents, such that they are relatively shelf-stable and resistant to microbial or fungal deterioration or contamination. Also preferred are dry food compositions which are extruded food products, such as pet foods, or snack foods for either humans or companion animals.

The compositions may also comprise one or more fiber sources. The term “fiber” includes all sources of “bulk” in the food whether digestible or indigestible, soluble or insoluble, fermentable or no fermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, fructooligosaccharide, pectin, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present disclosure to the immune system of an animal.

In other embodiments, the compositions further comprise prebiotics, probiotics, or a combination thereof. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics.

Prebiotics are non-digestible food ingredients that beneficially affect host health by selectively stimulating the growth and/or activity of bacteria in the colon. Prebiotics include fructooligosaccharides (FOS), xylooligosaccharides (XOS), galactooligosaccharides (GOS), and mannooligosaccharides (typically for non-human foods such as petfoods). The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes.

Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophillus, Bifidobacteria, Lactococcus and Pediococcus. Beneficial species include Enterococcus and Saccharomyces species.

The amount of probiotics and prebiotics to be administered to the animal is determined by the skilled artisan based upon the type and nature of the prebiotic and probiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day.

Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal.

The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package.

In a preferred embodiment, the food compositions comprise a macronutrient composition suitable for the type of food being designed. In one embodiment, the food composition has about 20 to 32% protein, about 30 to 50% carbohydrate, about 5% to 20% fat, and about 15% to 25% moisture. In another embodiment, the food composition is a pet food composition such as a premium or super-premium pet food composition. In one embodiment, the pet food is formulated for canines and has a protein content of about 20-30%, preferably about 24-28%, and more preferably about 25-27%. In one embodiment, the protein content of a dog food composition is about 26% by weight. In another embodiment, the formulation is for felines and has a protein content of about 35-45%, preferably about 37-42%, and more preferably about 39-41%. In one embodiment, the protein content of a cat food composition is about 40%. In a preferred embodiment, the composition is a food product comprising about 15% to about 50% protein, about 5% to about 40% fat, about 5% to about 10% ash content, and having a moisture content of about 5% to about 20%.

In some embodiments, the food composition is a wet food, such as a canned food, frozen food, or fresh food product. In some embodiments, the food composition is shelf stable. In other embodiments, it must be refrigerated. In other embodiments, the food composition is an intermediate moisture product or a dry food product as described above.

In some embodiments, the composition is administered to the animal in conjunction with one or more cognitive drugs in an amount effective for enhancing cognitive and related functions as defined herein. In a preferred embodiment, the composition is administered to the animal on a daily basis, preferably in a single dose.

In certain embodiments, the animal is a healthy aging animal. In others, the animal has a phenotype associated with age-related cognitive impairment. For example, when compared to a control animal not having the phenotype, the animal may have a phenotype that includes one or more of decreased ability to recall, short-term memory loss, decreased learning rate, decreased capacity for learning, decreased problem solving skills, decreased attention span, decreased motor performance, increased confusion, or dementia (Alzheimer's in humans or its equivalent in other animals).

In some embodiments, the composition comprises a combination of nutrients, such as a combination of spermidine and ellagic acid, or a combination of docosahexaenoic acid (DHA) and curcumin, in an amount effective for improving one or more social behaviors. In a preferred embodiment, the animal is a companion animal.

In some embodiments, the method achieves at least one result selected from the group consisting of: (a) reducing or preventing a decline of social interaction in an animal (e.g., to ensure that an aging animal remains involved in playtime, participates in group activities, interacts with caregivers, and the like); (b) reducing or preventing age-related behavioral changes in an animal (e.g., one or more of forgetfulness; disorientation; reduced social interaction; changes in sleep and wake habits, particularly an increase in nighttime activity; loss of “housetraining” that results in changes in urination and defecation locations and patterns; confusion; frustration; change in temperament, such as agitation and aggression; pacing; and wandering (such changes have been noted in response to cognitive declines in humans and other animals); (c) increasing trainability of an animal, for example to permit the animal to learn a task or obey verbal, signal, or other commands more quickly than if training occurred without using the composition; (d) maintaining optimal brain function in an animal, for example to slow the progression of mental decline in aging dogs; (e) facilitating learning and memory in an animal, for example to help an aging animal remember facts and understand instructions; (f) reducing memory loss in an animal; (g) reducing brain aging in an animal; (h) preventing or treating strokes in an animal, for example by reducing damage that results from strokes which is correlated to certain aspects of enhancing cognitive function, e.g., reducing memory loss; (i) preventing or treating dementia in an animal, for example Alzheimer's disease (AD) in humans, Canine Cognitive Dysfunction Syndrome (CCDS) in canines, or similar diseases in other animals, for example by reducing the effects of damage that results from the causes of dementia, e.g., amyloid deposits or deterioration of artery function; (j) maintaining mental clarity and alertness in an animal; (k) promoting the health and wellness of an animal; and (l) extending the prime for an animal.

Accordingly, in view of the preceding disclosures, an embodiment is a method of enhancing cognitive function and emotional health in a mammal, the method comprising administering a composition comprising at least one of (a) a combination of spermidine and ellagic acid or (b) a combination of docosahexaenoic acid (DHA) and curcumin to the mammal.

In some embodiments, the mammal is an ageing or elderly human or an ageing or elderly companion animal, preferably an elderly dog.

In some embodiments, the mammal has a phenotype associated with age-related cognitive impairment, and preferably the phenotype comprises one or more of decreased ability to recall, short-term memory loss, decreased learning rate, decreased capacity for learning, decreased problem solving skills, decreased attention span, decreased motor performance, increased confusion, or dementia, as compared to a control mammal not having the phenotype.

In some embodiments, the enhacing of emotional health comprises decreasing at least one of anxiety, stress, aggressivity, despair, lack of focus, or irritability and/or improving at least one of mood or social behavior.

In some embodiments, the administering uses at least one route selected from the group of oral, enteral, parenteral and intravenous injection.

In some embodiments, the composition has a form selected from the group consisting of a pet food, a dietary supplement, and a food product formulated for human consumption.

Another embodiment is a unit dosage form of a composition comprising at least one of (a) a combination of spermidine and ellagic acid or (b) a combination of docosahexaenoic acid (DHA) and curcumin, in an amount effective to enhance cognitive function and emotional health in a mammal who is administered the composition. In some embodiments, the mammal is an ageing or elderly human or an ageing or elderly companion animal, preferably an elderly dog.

Yet another embodiment is a method of making a composition for administration to a mammal, the method comprising adding at least one of (a) a combination of spermidine and ellagic acid or (b) a combination of docosahexaenoic acid (DHA) and curcumin to at least one other ingredient to form the composition. In some embodiments, the composition is formulated for administration by at least one route selected from the group of oral, enteral, parenteral and intravenous injection. In some embodiments, the at least one other ingredient is selected from the group consisting of protein, carbohydrate, lipid, vitamin, mineral, probiotic, prebiotic, and mixtures thereof. In some embodiments, the mammal is an ageing or elderly human or an ageing or elderly companion animal, preferably an elderly dog.

Another embodiment is a method comprising administering a composition comprising at least one of (a) a combination of spermidine and ellagic acid or (b) a combination of docosahexaenoic acid (DHA) and curcumin, wherein the composition is administered to a mammal in an amount effective to enhance cognitive function and emotional health in the mammal. In some embodiments, the composition is administered by at least one route selected from the group of oral, enteral, parenteral and intravenous injection. In some embodiments, the mammal is an ageing or elderly human or an ageing or elderly companion animal, preferably an elderly dog.

### Example

The following non-limiting example presents scientific data developing and supporting the inventive concepts disclosed herein.

Selected nutrient candidares were tested, specifically spermidine (polyamine), which can be provided by natural food sources such as wheat germ, soybean, cheese and poultry; resveratrol (stillbenoid) which can be provided by natural food sources such as Polygonum cuspidatum extract; ellagic acid (dilactone) which can be provided by natural food sources such as pomegranate extract and pomace; curcumin (curcuminoid) which can be provided by natural food sources such as Curcuma longa; EPA and DHA (omega-3 fatty acids) which can be provided by natural food sources such as fish oil; L-theanine (amino acid analog) which can be provided by natural food sources such as tea extract; beta-hydroxybutyrate (BHB-ketone); thymol; and isoflavones which can be provided by natural food sources such as soy.

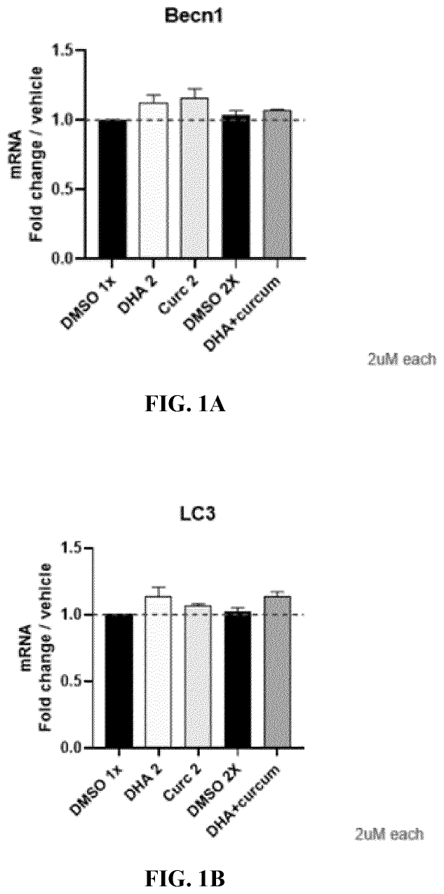

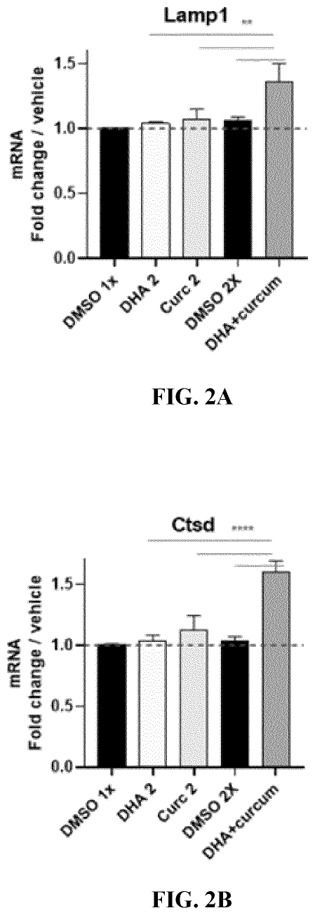

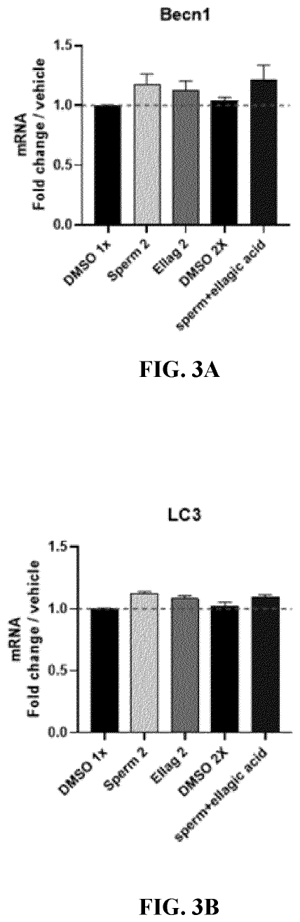

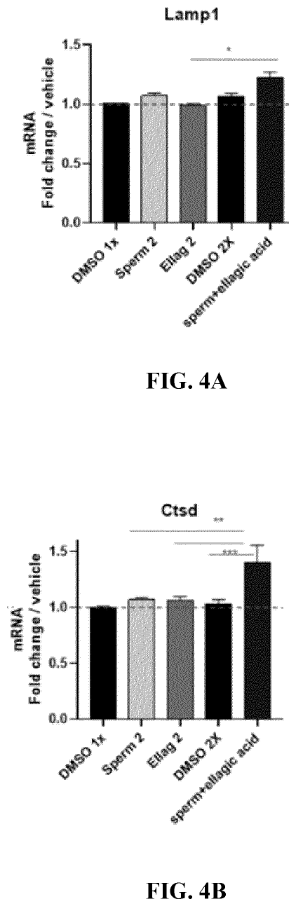

The results show there were no differences in the autophagy-related genes (FIGS. 1A and 1B) but significant synergy in lysosomal genes (FIGS. 2A and 2B) by the combination of DHA and curcumin. The results also show there were no differences in the autophagy-related genes (FIGS. 3A and 3B) but significant synergy in lysosomal genes (FIGS. 4A and 4B) by the combination of spermidine and ellagic acid.

These results are relevant when compared to literature. For example, Fu X. et al. (Experimental Neurology 2021) found that a molecule such as PF11 acting through TFEB increase Lamp1 and Ctsd mRNA in a similar manner as the two blends (DHA and curcumin, and spermidine and ellagic acid) and are neuroprotective. As another example, Huang et al. (CNS, Neuroscience and Therapeutics 2018) found that exercise activates lysosomal function in the brain through SIRT1-TFEB pathway more efficiently than trehalose.

As a summary of the scientific rationale:
Autophagy flux has been linked to synaptic function and psychiatric disorder (for a review see Tomoda T. et al, Biological Psychiatry 2019)
Autophagy flux and lysosomal activity are key regulators of the balanced excitatory and inhibitory transmission in the brain (see Sumitomo A. et al., Human Molecular Genetics 2018; Kuijpers M. et al., Neuron 2020; Hui K K. et al., Science Advances 2019; Lakhani R. et al., EMBO Molecular Medicine 2014)
Autophagy flux and lysosomal activity are key mediators of mood stabilizers and stress responses (Shu X. et al., Cell Death and Disease 2019; Gulbins A. et al., Molecular Psychiatry 2018; Gassen N C et al., Plos Medicine 2014; Limanaqi F. et al., Frontiers in Psychatry 2020;. Zhou et al., Progress in Neuro-Psychopharmacology & Biological Psychiatry 2015; Wang S. et al., Neuroscience Letters 2021)
Autophagy flux and lysosomal activity support brain and mental health by a plethora of mechanisms such as neurogenesis and others see above (Leeman D S. et al., Science 2018; Fleming A. et al., Trends in Neuroscience 2020).

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of enhancing cognitive function and emotional health in a mammal, the method comprising administering a composition comprising at least one of (a) a combination of spermidine and ellagic acid or (b) a combination of docosahexaenoic acid (DHA) and curcumin to the mammal.

2. The method of claim 1, wherein the mammal is an ageing or elderly human or an ageing or elderly companion animal.

3. The method of claim 1, wherein the mammal has a phenotype associated with age-related cognitive impairment.

4. The method of claim 1, wherein the enhancing of emotional health comprises decreasing at least one of anxiety, stress, aggressivity, despair, lack of focus, or irritability and/or improving at least one of mood or social behavior.

5. The method of claim 1, wherein the administering uses at least one route selected from the group consisting of oral, enteral, parenteral and intravenous injection.

6. The method of claim 1, wherein the composition has a form selected from the group consisting of a pet food, a dietary supplement, and a food product formulated for human consumption.

7. A unit dosage form of a composition comprising at least one of (a) a combination of spermidine and ellagic acid or (b) a combination of docosahexaenoic acid (DHA) and curcumin, in an amount effective to enhance cognitive function and emotional health in a mammal who is administered the composition.

8. The unit dosage form of claim 7, wherein the mammal is an ageing or elderly human or an ageing or elderly companion animal, preferably an elderly dog.

9. A method of making a composition for administration to a mammal, the method comprising adding at least one of (a) a combination of spermidine and ellagic acid or (b) a combination of docosahexaenoic acid (DHA) and curcumin to at least one other ingredient to form the composition.

10. (canceled)

11. The method of claim 9, wherein the at least one other ingredient is selected from the group consisting of protein, carbohydrate, lipid, vitamin, mineral, probiotic, prebiotic, and mixtures thereof.

12. The method of claim 9, wherein the mammal is an ageing or elderly human or an ageing or elderly companion animal, preferably an elderly dog.

13-15. (canceled)
